(19) United States
(12) Patent Application Publication
(10) Pub. No.: US 2024/0000205 A1
(43) Pub. Date: Jan. 4, 2024

(54) RING WEARABLE COVER WITH NON-DEFORMABLE CIRCUMFERENCE

(71) Applicant: Oura Health Oy, Oulu (FI)

(72) Inventors: Jose Julio Doval, Escondido, CA (US); Avi Halpern, Escondido, CA (US); Gary Watts, Oceanside, CA (US); Jonathan Watson, San Diego, CA (US); Kirt Alan Winter, San Diego, CA (US); Teemu Haverinen, Espoo (FI)

(21) Appl. No.: 17/855,098

(22) Filed: Jun. 30, 2022

(51) Int. Cl. A44C 9/00; G06F 3/01; G06F 1/16; H04W 4/80

(52) U.S. Cl. CPC A44C 9/0053; A44C 9/0023; G06F 3/014; G06F 1/163; H04W 4/80

(57) ABSTRACT

Systems and devices for a ring wearable cover with non-deformable circumference are described. The removable cover for a wearable ring device may include a ring-shaped surface configured to extend around a full circumference of the wearable ring device when the removable cover is in a mounted state on the wearable ring device and one or more mounting features that are disposed on the ring-shaped surface. The one or more mounting features may be configured to interact with a surface of the wearable ring device to lock the removable cover onto the wearable ring device when the removable cover is in the mounted state on the wearable ring device. In some cases, a diameter of the removable cover is unchanged while the removable cover transitions from the mounted state on the wearable ring device to an unmounted state off of the wearable ring device.

### FIELD OF TECHNOLOGY

The following relates to wearable devices and data processing, including a ring wearable cover with a non-deformable circumference.

### BACKGROUND

Some wearable devices may be configured to collect data from users including temperature data, heart rate data, and the like. Many users have a desire for more insight regarding their physical health. However, a user's movement and/or activity may displace or damage components of some wearable devices due to contact between the wearable device and different surfaces in the environment.

### BRIEF DESCRIPTION OF THE DRAWINGS

FIG. 1 illustrates an example of a system that supports ring wearable covers with a non-deformable circumference in accordance with aspects of the present disclosure.

FIG. 2 illustrates an example of a system that supports ring wearable covers with a non-deformable circumference in accordance with aspects of the present disclosure.

FIG. 3A illustrates an example of a ring wearable system in an unmounted state that supports ring wearable covers with a non-deformable circumference in accordance with aspects of the present disclosure.

FIG. 3B illustrates an example of a ring wearable system in a mounted state that supports ring wearable covers with a non-deformable circumference in accordance with aspects of the present disclosure.

FIG. 4A illustrates an example of a ring wearable system in an unmounted state that supports ring wearable covers with a non-deformable circumference in accordance with aspects of the present disclosure.

FIG. 4B illustrates an example of a ring wearable system in a mounted state that supports ring wearable covers with a non-deformable circumference in accordance with aspects of the present disclosure.

FIG. 5A illustrates an example of a ring wearable system in an unmounted state that supports ring wearable covers with a non-deformable circumference in accordance with aspects of the present disclosure.

FIG. 5B illustrates an example of a ring wearable system in a mounted state that supports ring wearable covers with a non-deformable circumference in accordance with aspects of the present disclosure.

FIG. 6A illustrates an example of a ring wearable system in an unmounted state that supports ring wearable covers with a non-deformable circumference in accordance with aspects of the present disclosure.

FIG. 6B illustrates an example of a ring wearable system in a mounted state that supports ring wearable covers with a non-deformable circumference in accordance with aspects of the present disclosure.

### DETAILED DESCRIPTION

Some wearable devices may be configured to collect data from users associated with movement and other activities. For example, some wearable devices may be configured to continuously acquire physiological data associated with a user including temperature data, heart rate data, and the like. As such, some wearable devices may be configured to house one or more sensors configured to acquire physiological data from a user.

Moreover, external surfaces of the wearable device may be damaged (e.g., bent, scratched, etc.) from surfaces that come in contact with the wearable device. In some cases, the surfaces that may contact the wearable device through the user's movement may include, but are not limited to, rough surfaces, hard surfaces, scratchy surfaces, or a combination thereof. For example, a user's movement may displace or damage components of some wearable devices, which may detrimentally affect the ability of the wearable device to efficiently and accurately acquire physiological data and increase an amount of the noise in the signal. Taken together, these issues with wearable devices may result in damage to a surface of the wearable device and, in some cases, inaccurate physiological data readings, which may lead to a distorted picture of the user's overall health, as well as increased power consumption and decreased battery life. In some implementations, the wearable devices may lack individuality such that the wearable devices may include a similar design and aesthetic that lacks personalization from one wearable device to another. As such, techniques for protecting the wearable device and adding personalized design may be desired.

Accordingly, to facilitate improved health monitoring, aspects of the present disclosure are directed to a removable cover for a ring wearable device. As described herein, the removable cover may be configured to mount or otherwise lock onto the wearable device such that the diameter of the removable cover is unchanged while the removable cover transitions from the mounted state on the wearable device to an unmounted state off of the wearable device. The removable cover may include a ring-shaped surface configured to extend around a full circumference of the wearable device when the removable cover is in a mounted state on the wearable ring device. In such cases, the ring-shaped surface is configured to extend around a full circumference of the removable cover when the removable cover is in an unmounted state off of the wearable ring device.

The removable cover may include one or more mounting features that are disposed on the ring-shaped surface. The one or more mounting features may be configured to interact with a surface or feature of the wearable ring device to lock the removable cover onto the wearable ring device when the removable cover is in the mounted state on the wearable ring device. The one or more mounting features may be an example of one or more protrusions in the ring-shaped surface, one or more cavities that extend at least partially through the ring-shaped surface, one or more tabs extending from the ring-shaped surface, one or more curved protrusions extending from the ring-shaped surface, or a combination thereof.

A material of the removable cover may be configured to maintain the diameter of the removable cover in an unchanged state while the removable cover transitions from the mounted state on the wearable ring device to the unmounted state off of the wearable ring device. In some cases, the removable cover may include a variety of materials, colors, designs, functionality, or a combination thereof. In such cases, the removable cover may be mounted onto the wearable device, via the one or more mounting features, to ensure a secure fit maintained during daily use and provide added protection to the components of the wearable device, added personalized design and aesthetics to the wearable device, or both.

The removable cover with a non-deformable circumference may protect the wearable device such that one or more antenna elements disposed within the wearable device may continue to wirelessly couple one or more components of the wearable device with a user device, the ring-shaped surface, or both, thereby decreasing an amount of noise in the signal and increasing the efficiency and accuracy of the signal. By implementing the removable cover with one or more mounting features on the surface of the wearable device, techniques described herein may lead to more accurate physiological data measurements.

Aspects of the disclosure are initially described in the context of systems supporting physiological data collection from users via wearable devices. Additional aspects of the disclosure are described in the context of example rings and ring covers. Although many of the examples of a wearable device depicted herein are ring-shaped wearable devices, it should be understood that the ring covers described herein may also be used with wearable devices of other form factors such as watches, patches, and the like.

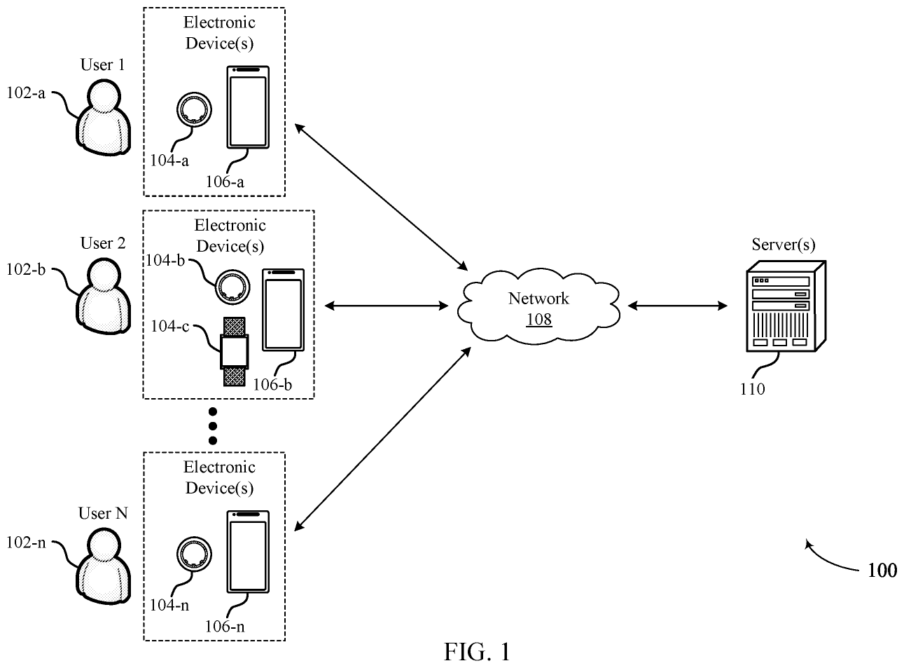

FIG. 1 illustrates an example of a system 100 that supports ring wearable cover with non-deformable circumference in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user's finger. In particular, a ring 104 may utilize one or more LEDs (e.g., red LEDs, green LEDs) that emit light on the palm-side of a user's finger to collect physiological data based on arterial blood flow within the user's finger. In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time during which a user 102 is asleep, and classify periods of time during which the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time during which the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g. in a hypothetical culture with 12 day “weeks”, 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for a removable cover for a wearable ring device (e.g., wearable device 104). The removable cover may include a ring-shaped surface configured to extend around a full circumference of the wearable device 104 when the removable cover is in a mounted state on the wearable device 104. In some cases, the removable cover may include one or more mounting features that are disposed on the ring-shaped surface and that are configured to interact with a surface of the wearable device 104 to lock the removable cover onto the wearable device 104 when the removable cover is in the mounted state on the wearable device 104. For example, a diameter of the removable cover is unchanged while the removable cover transitions from the mounted state on the wearable device 104 to an unmounted state off of the wearable device 104.

The wearable device 104 may surround a finger, wrist, ankle, or the like, of a user 102. The wearable device 104 may take measurements via the one or more sensors (e.g., heart rate measurements, oxygen saturation measurements (SpO2), temperature, sleep measurements, and the like). In some cases, materials may affect the optical behavior of sensor channels that take measurements. For example, sweat, dirt, water, other liquids, and the like may interfere with a signal quality associated with the physiological data acquired by the one or more sensors and result in inaccurate measurements. In such cases, the wearable device 104 may include a removable cover to protect an external surface of the wearable device 104 from materials and external forces while adding personalized design and aesthetics to the wearable device 104, as described herein.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

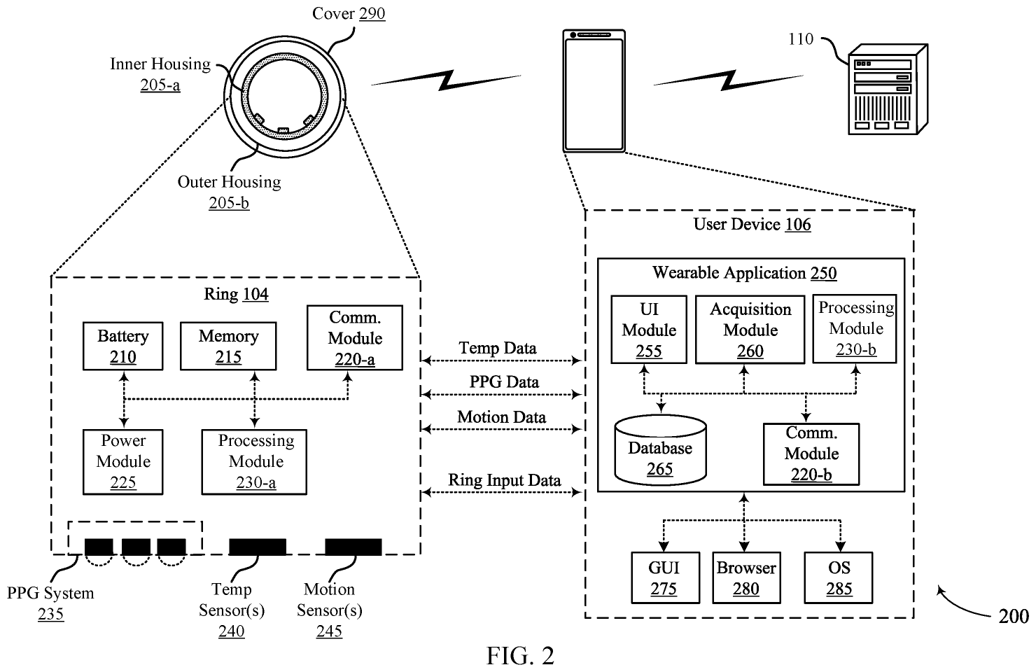

FIG. 2 illustrates an example of a system 200 that supports ring wearable cover with non-deformable circumference in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels, and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using a clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or which supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during 104 charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during 104 charging, and under voltage during 104 discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during 104 exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 in which the optical receiver(s) receive transmitted light that is reflected through the region of the user's finger. In some implementations, the PPG system 235 may be configured as a transmissive PPG system 235 in which the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBls. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BMl160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during 104 portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time in which the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the system 200 may support techniques for a ring wearable cover 290 with a non-deformable circumference. The removable cover 290 for a wearable ring device (e.g., wearable device 104) may include a ring-shaped surface configured to extend around a full circumference of the wearable ring device when the removable cover 290 is in a mounted state on the wearable ring device and one or more mounting features that are disposed on the ring-shaped surface. The one or more mounting features may be configured to interact with a surface or feature of the wearable ring device to lock the removable cover 290 onto the wearable ring device when the removable cover 290 is in the mounted state on the wearable ring device. In some cases, a diameter of the removable cover 290 is unchanged while the removable cover 290 transitions from the mounted state on the wearable ring device to an unmounted state off of the wearable ring device. For example, the removable cover 290 may include a non-deformable circumference.

Techniques described herein support a wearable ring device, such as a wearable device 104 as described with reference to FIG. 1. For example, a wearable device 104 may include an inner housing 205-a configured to house one or more sensors configured to acquire physiological data from a user 102 and an outer housing 205-b configured to house the inner housing 205-a. The one or more sensors may take physiological measurements from the user (e.g., temperature sensors, additional LED-PD sensors used for measuring heart rate, oxygen saturation, one or more sensors that a device may use to detect whether a user is asleep, or the like). In some cases, the one or more sensors are configured to acquire the physiological data from the user 102 based on arterial blood flow. In some implementations, the one or more sensors are configured to acquire the physiological data (e.g., including PPG data) from the user 102 based on blood flow that is diffused into the microvascular bed of skin with capillaries and arterioles.

The removable cover 290 may be configured to house the outer housing 205-b of the wearable device 104. As described in more detail herein, the ring-shaped surface of the removable cover 290 may include a material configured to maintain the diameter of the removable cover 290 in an unchanged state while the removable cover 290 transitions from the mounted state on the wearable ring device to the unmounted state off of the wearable ring device. In such cases, the ring-shaped surface extends three hundred and sixty degrees around the removable cover 290 relative to an axis of the removable cover 290 in the mounted state and the unmounted state. In some examples, the ring-shaped surface may be configured to interface with the surface of the wearable ring device (e.g., wearable device 104) such that one or more antenna elements disposed within the wearable ring device wirelessly couple one or more components of the wearable ring device with a user device, the ring-shaped surface of the removable cover 290, or both.

While much of the present disclosure describes the removable covers 290 in the context of a wearable ring cover for a wearable ring device, aspects of the present disclosure may additionally or alternatively be implemented in the context of other wearable devices. For example, in some implementations, the removable covers 290 described herein may be implemented in the context of other wearable devices, such as bracelets, watches, necklaces, piercings, and the like. For example, the wearable device 104 may surround a finger, wrist, ankle, or the like of a user. In some cases, the wearable device 104, the removable cover 290, or both may include a flat surface as opposed to a ring-shaped surface, as described herein.

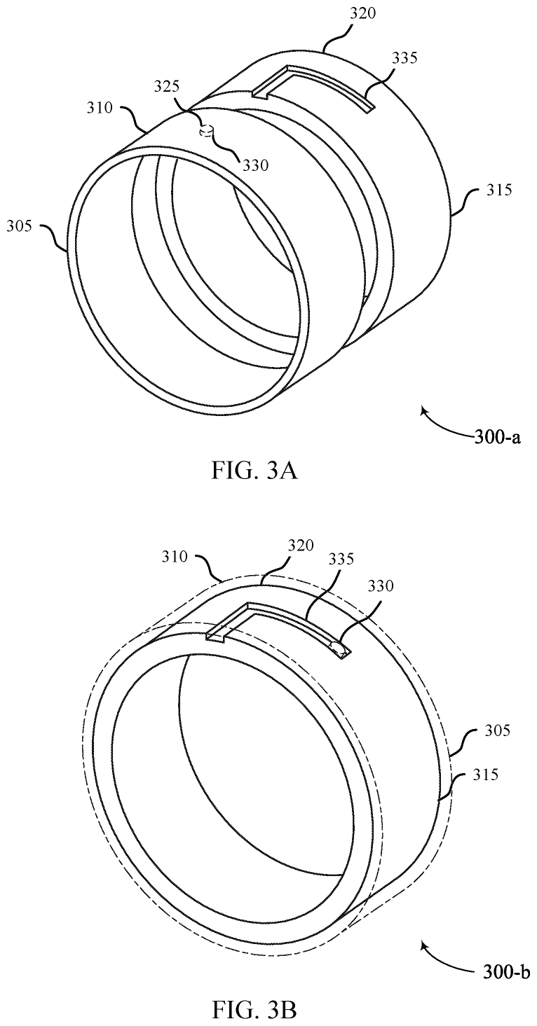

FIG. 3A illustrates an example of a ring wearable system 300-a in an unmounted state that supports a ring wearable cover with non-deformable circumference in accordance with aspects of the present disclosure. The ring wearable system 300-a may implement, or be implemented by, aspects of the system 100, system 200, or both. For example, ring wearable system 300 may illustrate examples of removable covers and wearable devices as described with reference to FIGS. 1 and 2. Specifically, the ring wearable system 300 may illustrate an orientation of a wearable ring device and removable cover on a user's finger. Although the wearable device and removable cover are illustrated as rings in FIG. 3, it may be any example of a wearable device (e.g., a watch, a necklace, and the like).

The removable cover 305 in ring wearable system 300 may include a ring-shaped surface 310. The ring-shaped surface 310 may be configured to extend a full circumference of the removable cover 305 when the removable cover 305 is in an unmounted state off of the wearable ring device 315. For example, the ring-shaped surface 310 may extend three hundred and sixty degrees around the removable cover 305 relative to an axis of the removable cover 305 in the unmounted state. The ring-shaped surface 310 may be an example of an outer surface or an inner surface of the removable cover 305.

The wearable ring device 315 in the ring wearable system 300 may include a surface 320. In some cases, the surface 320 may be an example of an outer surface of the wearable ring device 315.

In some examples, the removable cover 305 in ring wearable system 300 may include one or more mounting features 325 that are disposed on the ring-shaped surface 310. In some cases, the one or more mounting features 325 may include one or more protrusions 330 in the ring-shaped surface 310 extending outward away from the ring-shaped surface 310 (i.e., toward the center of the removable cover 305 and toward the surface 320 of the wearable device 315). For example, the removable cover 305 may include a single protrusion 330 in the ring-shaped surface 310 that is adhered to the ring-shaped surface 310 such that the protrusion 330 extends away from the ring-shaped surface 310 to a predetermined height. The protrusion 330 may be an example of a circular component positioned on the ring-shaped surface 310. In other examples, the protrusion 330 may be an example of any shaped component that may include, but is not limited to, rectangular, square, triangular, and the like.

The ring-shaped surface 310 may be formed from a material that may maintain a constant diameter of the removable cover 305 while the removable cover 305 transitions from the unmounted state off of the wearable ring device 315 to the mounted state on the wearable ring device 315. In such cases, the ring-shaped surface 310 may include a material configured to maintain the diameter of the removable cover 305 in an unchanged state while the removable cover 305 transitions from the mounted state on the wearable ring device 315 to the unmounted state off of the wearable ring device 315, and vice versa.

For example, the material may include, but is not limited to, a metallic material, an opaque material, a plastic material, or a combination thereof. In some cases, the ring-shaped surface 310 may be an example of an outer opaque shell. The protrusion 330 may include a same material as the ring-shaped surface 310. In other examples, the protrusion 330 may include a different material as the ring-shaped surface 310.

The surface 320 of the wearable ring device 315 may include one or more depressions 335 integrated into the surface 320 of the wearable ring device 315. The one or more depressions 335 may extend through a portion of the surface 320 of the wearable ring device 315. In such cases, the one or more depressions 335 may extend inward towards the surface 320 of the wearable ring device 315 such that the one or more depressions 335 may extend most of the way through the surface 320 of the wearable ring device 315 to a predetermined depth. The one or more depressions 335 may be an example of one or more channels integrated into the surface 320 of the wearable ring device 315. In some cases, the one or more depressions 335 may be an example of one or more cavities integrated into the surface 320.

The one or more mounting features 325 may be configured to interact with the surface 320 of the wearable ring device 315. For example, the protrusion 330 of the removable cover 305 may be configured to interact with the depression 335 of the wearable ring device 315. In such cases, the protrusion 330 is configured to interface with the depression 335 integrated into the surface 320 of the wearable ring device 315 to maintain the wearable ring device 315 in a defined position within the removable cover 305 in the mounted state, as described herein.

FIG. 3B illustrates an example of a ring wearable system 300-b in a mounted state that supports a ring wearable cover with a non-deformable circumference in accordance with aspects of the present disclosure. The protrusion 330 may be configured to interact with the surface 320 of the wearable ring device 315 to lock the removable cover 305 onto the wearable ring device 315 when the removable cover 305 is in the mounted state on the wearable ring device 315. For example, the protrusion 330 may be configured to interact with the depression 335 to lock the removable cover 305 onto the wearable ring device 315.

In such cases, the protrusion 330 in the ring-shaped surface 310 may extend toward the surface 320 of the wearable ring device 315 to fit within the depression 335 of the wearable ring device 315. As the removable cover 305 transitions from the unmounted state off of the wearable ring device 315, as described with reference to FIG. 3A, to the mounted state on the wearable ring device 315, the protrusion 330 may be positioned adjacent to the depression 335 such that the protrusion 330 aligns with a channel of the depression 335. The protrusion 330 may be sized to fit within the depression 335, and the wearable ring device 315 may be sized to fit within the removable cover 305.

The removable cover 305 may be advanced forward onto and over the wearable ring device 315 such that the protrusion 330 enters a channel of the depression 335. The protrusion 330 may be advanced along a first channel of the depression 335 until the protrusion 330 reaches an end of the first channel. The protrusion 330 may be advanced along a second channel of the depression 335 perpendicular to the first channel until the protrusion 330 reaches an end of the second channel. In such cases, the removable cover 305 is locked onto the wearable ring device 315 in the mounted state.

A constant diameter of the removable cover 305 may be unchanged while the removable cover 305 transitions from the unmounted state off of the wearable ring device 315 to the mounted state on the wearable ring device 315, and vice versa. For example, the diameter of the removable cover 305 may be unchanged during the transition between the unmounted state off of the wearable ring device 315 and the mounted state on the wearable ring device 315. The ring-shaped surface 310 may be configured to extend around a full circumference of the wearable ring device 315 when the removable cover 305 is in a mounted state on the wearable ring device 315. For example, the ring-shaped surface 310 may extend three hundred and sixty degrees around the removable cover 305 relative to an axis of the removable cover 305 in the mounted state.

As the removable cover 305 transitions from the mounted state on the wearable ring device 315, as described with reference to FIG. 3B, to the unmounted state off of the wearable ring device 315, the removable cover 305 may be turned sideways around the wearable ring device 315 (e.g., perpendicular) relative to the axis of the removable cover 305 such that the protrusion 330 is advanced along the second channel of the depression 335 until it reaches an end of the second channel. The protrusion 330 may then be advanced along the first channel of the depression 335 perpendicular to the second channel until the protrusion 330 exits the depression 335. In such cases, the removable cover 305 is unlocked from the wearable ring device 315, and the removable cover 305 is in the unmounted state off of the wearable ring device 315.

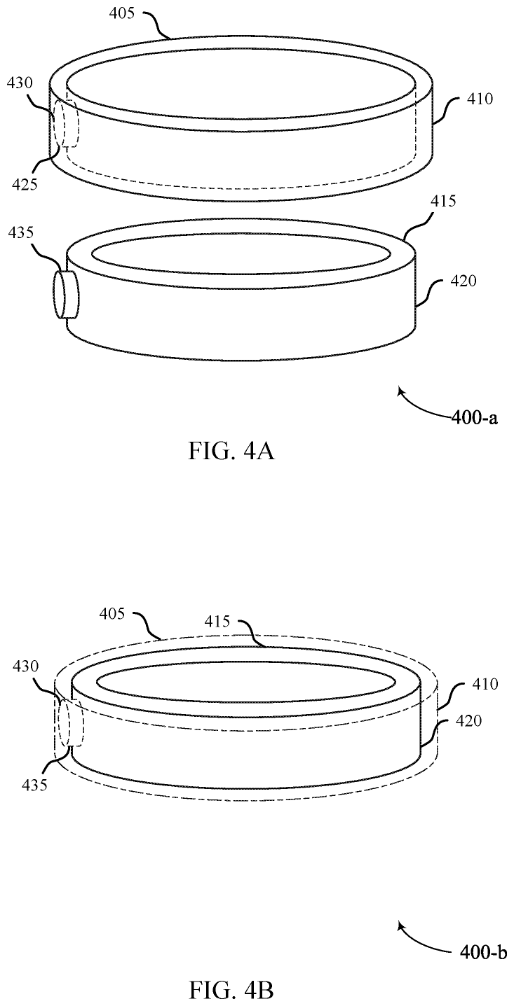

FIG. 4A illustrates an example of a ring wearable system 400-a in an unmounted state that supports a ring wearable cover with a non-deformable circumference in accordance with aspects of the present disclosure. The ring wearable system 400-a may implement, or be implemented by, aspects of the system 100, system 200, ring wearable system 300, or a combination thereof. For example, ring wearable system 400 may illustrate examples of the removable cover and the wearable ring device as described with reference to FIGS. 1-3. Specifically, the ring wearable system 400 may illustrate an orientation of a wearable ring device and removable cover on a user's finger. Although the wearable device and removable cover are illustrated as rings in FIG. 4, the wearable device and/or removable cover may be any example of a wearable device (e.g., a watch, a necklace, and the like).

As described with reference to FIG. 3, the removable cover 405 in ring wearable system 400 may include a ring-shaped surface 410 that is configured to extend a full circumference (e.g., three hundred and sixty degrees) of the removable cover 405 when the removable cover 405 is in an unmounted state off of the wearable ring device 415. In some cases, the ring-shaped surface 410 may be an example of an outer surface or an inner surface of the removable cover 405. The wearable ring device 415 in the ring wearable system 400 may include a surface 420, which may be an example of an outer surface of the wearable ring device 415.

The removable cover 405 in ring wearable system 400 may include one or more mounting features 425 that are disposed on the ring-shaped surface 410. In some cases, the one or more mounting features 425 may include one or more cavities 430 in the ring-shaped surface 410 that extend at least partially through the ring-shaped surface 410. For example, the removable cover 405 may include a single cavity 430 that extends inward towards the center of the removable cover 405. The cavity 430 may extend partially through a thickness of the removable cover 405 or all the way through the thickness of the removable cover 405.

The cavity 430 may be shaped as a circle, oval, rectangle, square, triangle, and the like. In some examples, the cavity 430 may be an example of a recess formed into the ring-shaped surface 410. In other examples, the one or more mounting features 425 may include one or more holes in the ring-shaped surface 410 that extend fully through the ring-shaped surface 410.

As described with reference to FIG. 3, the ring-shaped surface 410 may be formed from a material that may maintain a constant diameter of the removable cover 405 while the removable cover 405 transitions from the unmounted state off of the wearable ring device 415 to the mounted state on the wearable ring device 415, and vice versa. In such cases, the material of the ring-shaped surface 410 may be an example of the material of the ring-shaped surface as described with reference to FIG. 3. In some examples, the cavity 430 may include a same material as the ring-shaped surface 410.

The surface 420 of the wearable ring device 415 may include one or more protrusions 435 integrated into the surface 420 of the wearable ring device 415. In some cases, the wearable ring device 415 may include a single protrusion 435 that is adhered to the surface 420 such that the protrusion 435 extends from surface 420 to a predetermined height. In some examples, the single protrusion 435 may be a component of the surface 420 formed in a single piece during a molding process. In such cases, the protrusion 435 may extend outward away from the surface 420 of the wearable ring device 415. In some cases, the protrusion 435 may be an example of a bump disposed onto the surface 420 of the wearable ring device 415, a spring loaded pin, or both. In some examples, the protrusion 435 may be an example of a circular component positioned on the surface 420. In other examples, the protrusion 435 may be an example of any shaped component that may include, but is not limited to, rectangular, square, triangular, and the like.

The one or more mounting features 425 may be configured to interact with the surface 420 of the wearable ring device 415. For example, the cavity 430 of the removable cover 405 may be configured to interact with the protrusion 435 of the wearable ring device 415. In such cases, the cavity 430 is configured to interface with protrusion 435 disposed on the surface 420 of the wearable ring device 415 to maintain the wearable ring device 415 in a defined position within the removable cover 405 in the mounted state, as described herein.

FIG. 4B illustrates an example of a ring wearable system 400-b in a mounted state that supports a ring wearable cover with a non-deformable circumference in accordance with aspects of the present disclosure. The cavity 430 may be configured to interact with the surface 420 of the wearable ring device 415 to lock the removable cover 405 onto the wearable ring device 415 when the removable cover 405 is in the mounted state on the wearable ring device 415. For example, the cavity 430 of the removable cover 405 may be configured to interact with the protrusion 435 of the wearable ring device 415 to lock the removable cover 405 onto the wearable ring device 415.

In such cases, the protrusion 435 in the surface 420 of the wearable ring device 415 may extend toward the ring-shaped surface 410 of the removable cover 405 to fit within the cavity 430. As the removable cover 405 transitions from the unmounted state off of the wearable ring device 415, as described with reference to FIG. 4A, to the mounted state on the wearable ring device 415, the protrusion 435 may be positioned adjacent to the cavity 430 such that the protrusion 435 aligns with the cavity 430. The protrusion 435 may be sized to fit within the cavity 430, and the wearable ring device 415 may be sized to fit within the removable cover 405.

The removable cover 405 may be advanced (e.g., slid) onto and over the wearable ring device 415 such that the protrusion 435 enters the cavity 430. For example, the protrusion 435 may be configured to compress inwards towards the surface 420 of the wearable ring device 415 as the removable cover 405 transitions from the unmounted state off of the wearable ring device 415 to the mounted state on the wearable ring device 415. As the protrusion 435 enters the cavity 430 of the removable cover 405, the protrusion 435 may be configured to compress outward away from the surface 420 of the wearable ring device 415 in the mounted state. In such cases, the removable cover 405 is locked onto the wearable ring device 415 in the mounted state.

As described with reference to FIG. 3, a constant diameter of the removable cover 405 may be unchanged while the removable cover 405 transitions from the unmounted state off of the wearable ring device 415 to the mounted state on the wearable ring device 415, and vice versa. The ring-shaped surface 410 may be configured to extend around a full circumference (e.g., three hundred and sixty degrees) of the wearable ring device 415 when the removable cover 405 is in a mounted state on the wearable ring device 415.

As the removable cover 405 transitions from the mounted state on the wearable ring device 415, as described with reference to FIG. 4B, to the unmounted state off of the wearable ring device 415, the removable cover 405 may slide off the wearable ring device 415 such that the protrusion 435 exits the cavity 430. The removable cover 405 may be advanced off the wearable ring device 415 until the protrusion 435 is unlocked from the cavity 430. For example, the protrusion 435 may be configured to compress inwards towards the surface 420 of the wearable ring device 415 as the removable cover 405 transitions from the mounted state on the wearable ring device 415 to the unmounted state off of the wearable ring device 415. As the protrusion 435 exits the cavity 430 of the removable cover 405, the protrusion 435 may be configured to compress inwards towards the surface 420 of the wearable ring device 415 during the transition. The protrusion 435 may be configured to compress outwards away from the surface 420 of the wearable ring device 415 as the protrusion 435 clears the ring-shaped surface 410 of the removable cover 405 and the removable cover 405 is in the unmounted state. In such cases, the removable cover 405 is unlocked from the wearable ring device 415, and the removable cover 405 is in the unmounted state off of the wearable ring device 415.

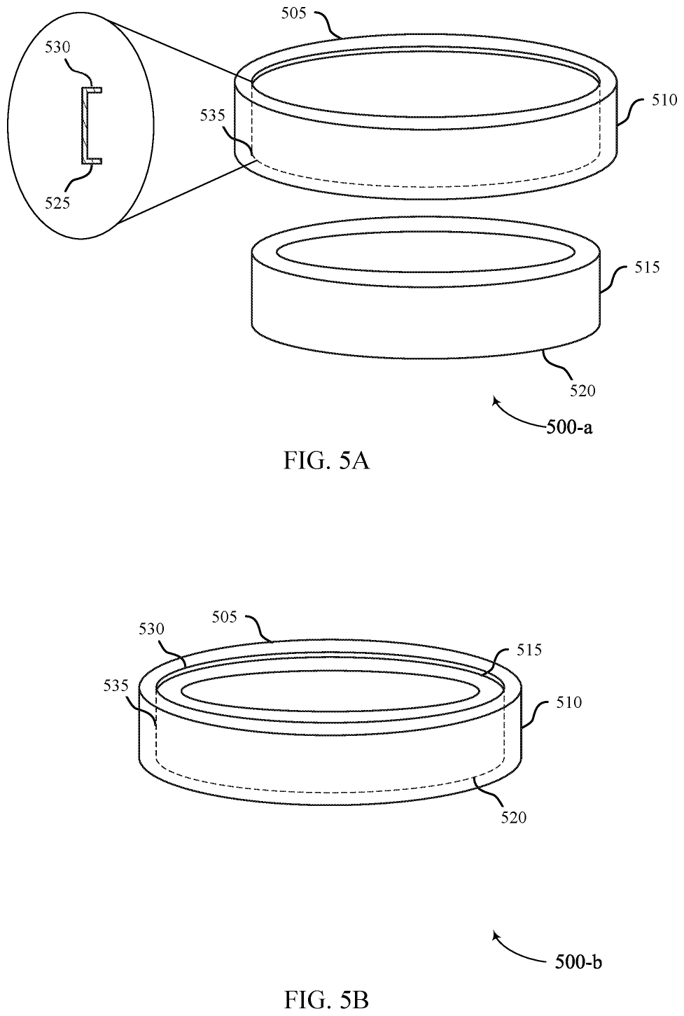

FIG. 5A illustrates an example of a ring wearable system 500-a in an unmounted state that supports a ring wearable cover with a non-deformable circumference in accordance with aspects of the present disclosure. The ring wearable system 500-a may implement, or be implemented by, aspects of the system 100, system 200, ring wearable system 300, ring wearable system 400, or a combination thereof. For example, ring wearable system 500 may illustrate examples of the removable cover and the wearable ring device as described with reference to FIGS. 1-4. Specifically, the ring wearable system 500 may illustrate an orientation of a wearable ring device and removable cover on a user's finger. Although the wearable device and removable cover are illustrated as rings in FIG. 5, the wearable device and/or removable cover may be any example of a wearable device (e.g., a watch or wrist-band, a necklace, and the like).

As described with reference to FIGS. 3 and 4, the removable cover 505 in ring wearable system 500 may include a ring-shaped surface 510 that is configured to extend a full circumference of the removable cover 505 when the removable cover 505 is in an unmounted state off of the wearable ring device 515. The ring-shaped surface 510 may be an example of an outer surface of the removable cover 505 or an inner surface of the removable cover 505. The wearable ring device 515 in the ring wearable system 500 may include a surface 520, which may be an example of an outer surface of the wearable ring device 515.

The ring-shaped surface 510 may include one or more circumferential edges 535. The one or more circumferential edges 535 may extend three hundred and sixty degrees around the removable cover 505 relative to an axis of the removable cover 505. For example, the ring-shaped surface 510 may include a first circumferential edge 535 around a circumference of the removable cover 505 and a second circumferential edge 535 opposite the first circumferential edge 535 around the circumference of the removable cover 505.

The removable cover 505 in ring wearable system 500 may include one or more mounting features 525 that are disposed on the ring-shaped surface 510. In some cases, the one or more mounting features 525 may include one or more curved protrusions 530 extending from the one or more circumferential edges 535 of the ring-shaped surface 510. In some cases, the one or more curved protrusions 530 may extend straight out from the one or more circumferential edges 535. For example, the removable cover 505 may include a first curved protrusion 530 and a second curved protrusion 530 opposite the first curved protrusion 530. The first curved protrusion 530 may extend from the first circumferential edge 535 a same distance as the second curved protrusion 530 extends from the second circumferential edge 535. In other examples, the first curved protrusion 530 may extend from the first circumferential edge 535 a different distance (e.g., less than or greater than) the second curved protrusion 530 extends from the second circumferential edge 535. In some cases, the first curved protrusion 530 may include a thickness different than the second curved protrusion 530 or the first curved protrusion 530 may include a thickness the same as the second curved protrusion 530.

The one or more curved protrusions 530 may be integrated into the ring-shaped surface 510 of the removable cover 505. The one or more curved protrusions 530 may extend outward away from the ring-shaped surface 510 of the removable cover. For example, the one or more curved protrusions 530 may extend outward away from the one or more circumferential edges 535. The one or more curved protrusions 530 may be an example of one or more locking grooves or lips disposed on the sides of the ring-shaped surface 510. In some cases, the one or more curved protrusions 530 may be an example of a spring loaded lip.

As described with reference to FIGS. 3 and 4, the ring-shaped surface 510 may be formed from a material that may maintain a constant diameter of the removable cover 505 while the removable cover 505 transitions from the unmounted state off of the wearable ring device 515 to the mounted state on the wearable ring device 515. In such cases, the material of the ring-shaped surface 510 may be an example of the material of the ring-shaped surface as described with reference to FIGS. 3 and 4. In some examples, the curved protrusions 530 may include a same material as the ring-shaped surface 510. In other examples, the curved protrusions 530 may include a different material as the ring-shaped surface 510.

The one or more mounting features 525 may be configured to interact with the surface 520 of the wearable ring device 515. For example, the one or more curved protrusions 530 of the removable cover 505 may be configured to interact with the wearable ring device 515 and maintain the wearable ring device 515 in a defined position within the removable cover 505 in the mounted state, as described herein.

FIG. 5B illustrates an example of a ring wearable system 500-b in a mounted state that supports ring wearable cover with non-deformable circumference in accordance with aspects of the present disclosure. The one or more curved protrusions 530 may be configured to interact with the surface 520 of the wearable ring device 515 to lock the removable cover 505 onto the wearable ring device 515 when the removable cover 505 is in the mounted state on the wearable ring device 515. For example, the one or more curved protrusions 530 of the removable cover 505 may be configured to interact with the sides of the wearable ring device 515 to lock the removable cover 505 onto the wearable ring device 515.

In such cases, the one or more curved protrusions 530 may extend toward the surface 520 of the wearable ring device 515 to snap or clip onto the wearable ring device 515. As the removable cover 505 transitions from the unmounted state off of the wearable ring device 515, as described with reference to FIG. 5A, to the mounted state on the wearable ring device 515, the removable cover 505 may be positioned adjacent to the wearable ring device 515 such that the one or more curved protrusions 530 aligns with the surface 520 of the wearable ring device 515. The wearable ring device 515 may be sized to fit within the removable cover 505.

The removable cover 505 may be advanced (e.g., slid) onto and over the wearable ring device 515 such that the first curved protrusion 530 is configured to compress inwards towards the ring-shaped surface 510 as the removable cover 505 transitions from the unmounted state off of the wearable ring device 515 to the mounted state on the wearable ring device 515. As the removable cover 505 is slid over the wearable ring device 515, the first curved protrusion 530 may be compressed inwards until the first curved protrusion 530 clears the surface 520 of the wearable ring device 515. The curved protrusion 530 may then be configured to extend outward away from the ring-shaped surface 510 of the removable cover 505 in the mounted state. The second curved protrusion 530 opposite the first curved protrusion 530 may be configured to extend outward away from the ring-shaped surface 510 of the removable cover 505 in the mounted state such that the one or more curved protrusions 530 are snapped onto both sides of the wearable ring device 515. In such cases, the removable cover 505 is locked onto the wearable ring device 515 in the mounted state.

In some cases, the one or more mounting features 525 (e.g., one or more curved protrusions 530) are configured to maintain the wearable ring device 515 within the removable cover 505 in the mounted state based on friction between the ring-shaped surface 510 and the surface 520 of the wearable ring device 515. As described with reference to FIGS. 3 and 4, a diameter of the removable cover 505 may be unchanged while the removable cover 505 transitions from the unmounted state off of the wearable ring device 515 to the mounted state on of the wearable ring device 515, and vice versa. The ring-shaped surface 510 may be configured to extend around a full circumference of the wearable ring device 515 when the removable cover 505 is in a mounted state on the wearable ring device 515.

As the removable cover 505 transitions from the mounted state on the wearable ring device 515, as described with reference to FIG. 5B, to the unmounted state off of the wearable ring device 515, the removable cover 505 may slide off the wearable ring device 515 such that the first curved protrusion 530 unsnaps from the wearable ring device 515. For example, the first curved protrusion 530 may be configured to compress inwards towards the ring-shaped surface 510 as the removable cover 505 transitions from the mounted state on the wearable ring device 515 to the unmounted state off of the wearable ring device 515.

As the first curved protrusion 530 clears the surface 520 of the wearable ring device 515, the first curved protrusion 530 may be configured to compress outwards away from the ring-shaped surface 510 of the removable cover 505 during the transition. In such cases, the removable cover 505 may be advanced off the wearable ring device 515 until the first curved protrusion 530 is unlocked from the surface 520 of the wearable ring device 515. For example, the removable cover 505 is unlocked from the wearable ring device 515, and the removable cover 505 is in the unmounted state off of the wearable ring device 515.

In some cases, the one or more mounting features 525 (e.g., one or more curved protrusions 530) may be removed from the removable cover 505. In such cases, the removable cover 505 transition to the mounted state on the wearable device 515 by using a forming press to press the removable cover 505 onto the wearable device 515. For example, a press fit, an interference fit, a force fit, or a combination thereof may be formed between the removable cover 505 and the wearable device 515 such that the removable cover 505 may be tightly fit around the wearable device 515.

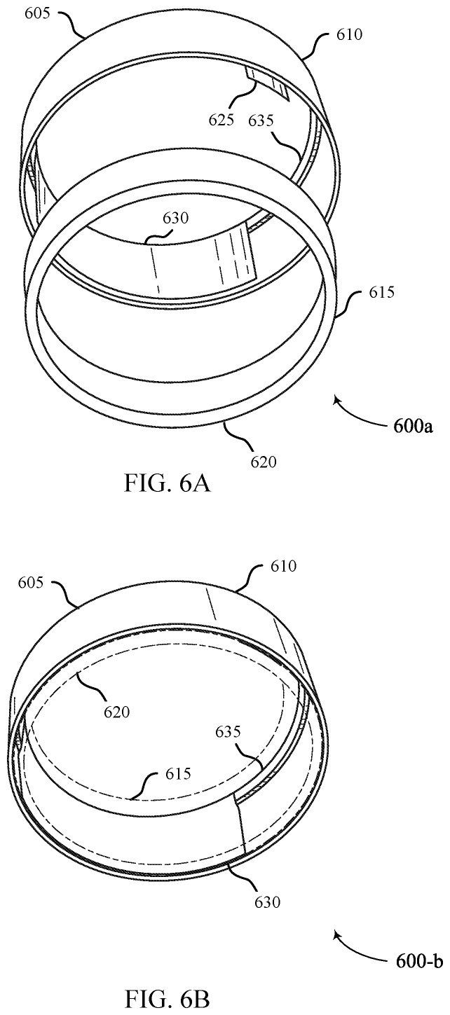

FIG. 6A illustrates an example of a ring wearable system 600-a in an unmounted state that supports a ring wearable cover with a non-deformable circumference in accordance with aspects of the present disclosure. The ring wearable system 600-a may implement, or be implemented by, aspects of the system 100, system 200, ring wearable system 300, ring wearable system 400, ring wearable system 500, or a combination thereof. For example, ring wearable system 600 may illustrate examples of the removable cover and the wearable ring device as described with reference to FIGS. 1-5. Specifically, the ring wearable system 600 may illustrate an orientation of a wearable ring device and removable cover on a user's finger. Although the wearable device and removable cover are illustrated as rings in FIG. 6, it may be any example of a wearable device (e.g., a watch, a necklace, and the like).

As described with reference to FIGS. 3-5, the removable cover 605 in ring wearable system 600 may include a ring-shaped surface 610 that is configured to extend three hundred and sixty degrees around the removable cover 605 relative to an axis of the removable cover 605 in the unmounted state. The wearable ring device 615 in the ring wearable system 600 may include a surface 620, which may be an example of an exterior surface.

The ring-shaped surface 610 may include an internal surface 635. In some examples, the internal surface 635 may be positioned opposite the ring-shaped surface 610. The internal surface 635 may extend three hundred and sixty degrees around the removable cover 605 relative to an axis of the removable cover 605. The removable cover 605 in ring wearable system 600 may include one or more mounting features 625 that are disposed on the ring-shaped surface 610. In some examples, the mounting features 625 may be dispersed equidistantly around the ring-shaped surface 610, or may be dispersed randomly around the ring-shaped surface. In some cases, the removable cover 605 may include a single mounting feature 625 that extends three hundred and sixty degrees around the removable cover 605 relative to an axis of the removable cover 605. In other examples, the removable cover 605 may include two or more mounting features 625.

The one or more mounting features 625 may include one or more tabs 630 extending from the internal surface 635 of the ring-shaped surface 610. In some cases, the one or more tabs 630 may extend straight out from the internal surface 635. In other examples, the one or more tabs 630 may extend towards or away from the internal surface 635 at an angle. For example, the removable cover 605 may include a first tab 630 and a second tab 630 opposite the first tab 630. The first tab 630 may extend from the internal surface 635 a same distance as the second tab 630 extends from the internal surface 635. In other examples, the first tab 630 may extend from the internal surface 635 a different distance (e.g., less than or greater than) the second tab 630 extends from the internal surface 635. In such cases, a height of the one or more tabs 630 may be the same or different. In some cases, the first tab 630 may include a thickness different from the second tab 630 or the first tab 630 may include a thickness the same as the second tab 630. A length of the first tab 630 may be different from a length of the second tab 630. In other examples, the length of the first tab 630 may be the same as the length of the second tab 630.

The one or more tabs 630 may be integrated into the internal surface 635 of the ring-shaped surface 610. The one or more tabs 630 may extend parallel to the ring-shaped surface 610 of the removable cover 605. For example, the one or more curved tabs 630 may extend parallel to the internal surface 635 of the ring-shaped surface 610. In the unmounted state, a gap may exist between the one or more tabs 630 and the ring-shaped surface 610. The one or more tabs 630 may be an example of one or more locking components disposed on the inside of the ring-shaped surface 610, one or more spring loaded tabs, or both.

In some examples, the one or more tabs 630 may be an example of one or more O-rings (e.g., circular protrusions) that extend three hundred and sixty degrees around the internal surface 635 of the removable cover 605 relative to an axis of the removable cover 605. In some cases, the wearable ring device 615 may include one or more depressions or channels configured to interface with the one or more O-rings of the removable cover 605. In other examples, the surface 620 of the wearable ring device 615 may include one or more O-rings that extend three hundred and sixty degrees around the surface 620 of the wearable ring device 615 relative to an axis of the wearable ring device 615.

As described with reference to FIGS. 3-5, the ring-shaped surface 610 may be formed from a material that may maintain a constant diameter of the removable cover 605 while the removable cover 605 transitions from the unmounted state off of the wearable ring device 615 to the mounted state on the wearable ring device 615. In such cases, the material of the ring-shaped surface 610 may be an example of the material of the ring-shaped surface as described with reference to FIGS. 3-5. In some examples, the one or more tabs 630 may include a same material as the ring-shaped surface 610. In other examples, the one or more tabs 630 may include a different material as the ring-shaped surface 610.

The one or more mounting features 625 may be configured to interact with the surface 620 of the wearable ring device 615. For example, the one or more curved tabs 630 of the removable cover 605 may be configured to interact with the wearable ring device 615 and maintain the wearable ring device 615 in a defined position within the removable cover 605 in the mounted state, as described herein.

FIG. 6B illustrates an example of a ring wearable system 600-b in a mounted state that supports a ring wearable cover with a non-deformable circumference in accordance with aspects of the present disclosure. The one or more tabs 630 may be configured to interact with the surface 620 of the wearable ring device 615 to lock the removable cover 605 onto the wearable ring device 615 when the removable cover 605 is in the mounted state on the wearable ring device 615. For example, the one or more tabs 630 of the removable cover 605 may be configured to interact with an internal surface of the wearable ring device 615 to lock the removable cover 605 onto the wearable ring device 615.

In such cases, the one or more tabs 630 may extend toward the surface 620 of the wearable ring device 615 to snap onto the wearable ring device 615. As the removable cover 605 transitions from the unmounted state off of the wearable ring device 615, as described with reference to FIG. 6A, to the mounted state on the wearable ring device 615, the removable cover 605 may be positioned adjacent to the wearable ring device 615 such that the one or more tabs 630 align with the surface 620 of the wearable ring device 615. The wearable ring device 615 may be sized to fit within the removable cover 605.

The removable cover 605 may be advanced (e.g., slid) onto and over the wearable ring device 615 such that the one or more tabs 630 are configured to compress inwards towards the ring-shaped surface 610 as the removable cover 605 transitions from the unmounted state off of the wearable ring device 615 to the mounted state on the wearable ring device 615. As the removable cover 605 is slid over the wearable ring device 615, the one or more tabs 630 are compressed inwards until a circumferential edge of the removable cover 605 aligns with a circumferential edge of the wearable ring device 615. The one or more tabs 630 may then be configured to extend outward away from the ring-shaped surface 610 of the removable cover 605 to form an interference fit with the surface 620 of the wearable ring device 615 in the mounted state. The one or more tabs 630 may be configured to extend outward away from the ring-shaped surface 610 of the removable cover 605 in the mounted state such that the one or more tabs 630 are snapped onto the wearable ring device 615. For example, in the mounted state, the gap may be minimized between the one or more tabs 630 and the ring-shaped surface 610. In such cases, the removable cover 605 is locked onto the wearable ring device 615 in the mounted state.

As described with reference to FIG. 5, the one or more mounting features 625 (e.g., one or more tabs 630) are configured to maintain the wearable ring device 615 within the removable cover 605 in the mounted state based on friction between the ring-shaped surface 610 and the surface 620 of the wearable ring device 515. As described with reference to FIGS. 3-5, a diameter of the removable cover 605 may be unchanged while the removable cover 605 transitions from the unmounted state off of the wearable ring device 615 to the mounted state on the wearable ring device 615, and vice versa. In such cases, the ring-shaped surface 610 may be configured to extend around a full circumference of the wearable ring device 615 when the removable cover 605 is in a mounted state on the wearable ring device 615.

As the removable cover 605 transitions from the mounted state on the wearable ring device 615, as described with reference to FIG. 6B, to the unmounted state off of the wearable ring device 615, the removable cover 605 may slide off the wearable ring device 615 such that the one or more tabs 630 unsnap from the wearable ring device 515. For example, the one or more tabs 630 may be configured to compress inwards towards the ring-shaped surface 610 as the removable cover 605 transitions from the mounted state on the wearable ring device 615 to the unmounted state off of the wearable ring device 615.

The one or more tabs 630 may clear the surface 620 of the wearable ring device 615 as the removable cover transitions to the unmounted state, and the one or more tabs 630 may be configured to extend outwards away from the ring-shaped surface 510 of the removable cover 505 such that the gap exists between the one or more tabs 630 and the ring-shaped surface 610. In such cases, the removable cover 605 may be advanced off the wearable ring device 615 until the one or more tabs 630 are unlocked from the surface 620 of the wearable ring device 615. For example, the removable cover 605 is unlocked from the wearable ring device 615, and the removable cover 605 is in the unmounted state off of the wearable ring device 615.

In some cases, the mounting features as described with reference to FIGS. 3-6 may be used as separate mounting features in each removable cover or as a combination of mounting features in a same removable cover. In some examples, the removable cover as described with reference to FIGS. 3-6 may include a magnet or magnetic material in the ring-shaped surface, the one or more mounting features, or both.

It should be noted that the methods described above describe possible implementations, and that the operations and the steps may be rearranged or otherwise modified and that other implementations are possible. Furthermore, aspects from two or more of the methods may be combined.

An apparatus is described. The apparatus may include a ring-shaped surface configured to extend around a full circumference of the wearable ring device when the removable cover is in a mounted state on the wearable ring device and one or more mounting features that are disposed on the ring-shaped surface and that are configured to interact with a surface of the wearable ring device to lock the removable cover onto the wearable ring device when the removable cover is in the mounted state on the wearable ring device, wherein a diameter of the removable cover is unchanged while the removable cover transitions from the mounted state on the wearable ring device to an unmounted state off of the wearable ring device.

In some examples of the apparatuses, the one more mounting features may include one or more protrusions in the ring-shaped surface extending outward toward the surface of the wearable ring device, wherein the one or more protrusions may be configured to interface with one or more depressions integrated into the surface of the wearable ring device to maintain the wearable ring device in a defined position within the removable cover in the mounted state.

In some examples of the apparatuses, the one more mounting features may include one or more cavities that extend at least partially through the ring-shaped surface that may be configured to interface with one or more protrusions integrated into the surface of the wearable ring device and that extend outward towards the ring-shaped surface to maintain the wearable ring device in a defined position within the removable cover in the mounted state.

In some examples of the apparatuses, the one or more mounting features may be configured to compress inward towards the ring-shaped surface as the removable cover transitions from the unmounted state off the wearable ring device to the mounted state on the wearable ring device.

In some examples of the apparatuses, the one more mounting features may include one or more tabs extending from an internal surface of the ring-shaped surface and configured to maintain the wearable ring device within the removable cover in the mounted state.

In some examples of the apparatuses, the one more mounting features may include one or more curved protrusions extending from one or more circumferential edges of the ring-shaped surface and configured to maintain the wearable ring device within the removable cover in the mounted state.

In some examples of the apparatuses, the one or more mounting features may be configured to maintain the wearable ring device within the removable cover in the mounted state based on friction between the ring-shaped surface and the surface of the wearable ring device.

In some examples of the apparatuses, the ring-shaped surface comprises a material configured to maintain the diameter of the removable cover in an unchanged state while the removable cover transitions from the mounted state on the wearable ring device to the unmounted state off of the wearable ring device.

In some examples of the apparatuses, the ring-shaped surface extends three hundred and sixty degrees around the removable cover relative to an axis of the removable cover in the mounted state and the unmounted state.

In some examples of the apparatuses, the ring-shaped surface may be configured to interface with the surface of the wearable ring device such that one or more antenna elements disposed within the wearable ring device wirelessly couple one or more components of the wearable ring device with a user device, the ring-shaped surface, or both.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended FIGURES, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A removable cover for a wearable ring device comprising:
a ring-shaped surface configured to extend around a full circumference of the wearable ring device when the removable cover is in a mounted state on the wearable ring device; and
one or more mounting features that are disposed on the ring-shaped surface and that are configured to interact with a surface of the wearable ring device to lock the removable cover onto the wearable ring device when the removable cover is in the mounted state on the wearable ring device, wherein a diameter of the removable cover is unchanged while the removable cover transitions from the mounted state on the wearable ring device to an unmounted state off of the wearable ring device.

2. The removable cover of claim 1, wherein the one or more mounting features further comprise one or more protrusions in the ring-shaped surface extending outward toward the surface of the wearable ring device, wherein the one or more protrusions are configured to interface with one or more depressions integrated into the surface of the wearable ring device to maintain the wearable ring device in a defined position within the removable cover in the mounted state.

3. The removable cover of claim 1, wherein the one or more mounting features further comprise one or more cavities that extend at least partially through the ring-shaped surface that are configured to interface with one or more protrusions integrated into the surface of the wearable ring device and that extend outward towards the ring-shaped surface to maintain the wearable ring device in a defined position within the removable cover in the mounted state.

4. The removable cover of claim 1, wherein the one or more mounting features are configured to compress inward towards the ring-shaped surface as the removable cover transitions from the unmounted state off the wearable ring device to the mounted state on the wearable ring device.

5. The removable cover of claim 4, wherein the one or more mounting features further comprise one or more tabs extending from an internal surface of the ring-shaped surface and configured to maintain the wearable ring device within the removable cover in the mounted state.

6. The removable cover of claim 4, wherein the one or more mounting features further comprise one or more curved protrusions extending from one or more circumferential edges of the ring-shaped surface and configured to maintain the wearable ring device within the removable cover in the mounted state.

7. The removable cover of claim 1, wherein the one or more mounting features are configured to maintain the wearable ring device within the removable cover in the mounted state based on friction between the ring-shaped surface and the surface of the wearable ring device.

8. The removable cover of claim 1, wherein the ring-shaped surface comprises a material configured to maintain the diameter of the removable cover in an unchanged state while the removable cover transitions from the mounted state on the wearable ring device to the unmounted state off of the wearable ring device.

9. The removable cover of claim 1, wherein the ring-shaped surface extends three hundred and sixty degrees around the removable cover relative to an axis of the removable cover in the mounted state and the unmounted state.

10. The removable cover of claim 1, wherein the ring-shaped surface is configured to interface with the surface of the wearable ring device such that one or more antenna elements disposed within the wearable ring device wirelessly couple one or more components of the wearable ring device with a user device, the ring-shaped surface, or both.
